(19) United States
(12) Patent Application Publication
(10) Pub. No.: US 2022/0212029 A1
(43) Pub. Date: Jul. 7, 2022

(54) Therapy System, Bed System Comprising Same, and Method for Operating Therapy System

(71) Applicant: NF Co., Ltd, Busan (KR)

(72) Inventors: Sang Kone Lee, Busan (KR); Seung Chul Kim, Gyeonggi-do (KR)

(21) Appl. No.: 17/606,908

(22) PCT Filed: May 30, 2019
(86) PCT No.: PCT/KR2019/006521

§ 371 Date: Oct. 27, 2021

(30) Foreign Application Priority Data
May 13, 2019 (KR) 10-2019-0055371

(51) Int. Cl. A61N 5/06; A61M 16/10; A61B 5/16; A61B 5/25; A61M 21/00; A61M 21/02

(52) U.S. Cl. CPC A61N 5/0618; A61M 16/101; A61B 5/165; A61B 5/25; A61M 21/0094; A61M 2230/04; A61M 2021/0027; A61M 2021/0044; A61M 2021/0077; A61M 2205/051; A61M 21/02

(57) ABSTRACT

The present disclosure relates to a therapy system, a bed system including the same, and an operating method of the therapy system. The therapy system according to an embodiment of the present disclosure includes an electrocardiogram sensor to sense an electrocardiogram, a controller to determine a state step based on the electrocardiogram and to generate an oxygen control signal corresponding to the state step, an oxygen supply device to release oxygen having a concentration higher than an oxygen concentration in the atmosphere during an output time corresponding to the state step, based on the oxygen control signal. The controller may release the oxygen having a target concentration corresponding to the state step based on the oxygen control signal. Also, the controller may control a wavelength or a time of a light so as to correspond to the state step.

### TECHNICAL FIELD

The present disclosure relates to stress care, and more particularly, relates to a therapy system, a bed system including the same, and an operating method of the therapy system.

### BACKGROUND ART

With the development of industrial technology, modern people may work without distinction between day and night, may access a variety of information, and may face a new social structure. Due to a social structure getting more complex day by day, an excessive work, a study, or an interpersonal relationship, modern people are under higher stress than in the past. For this reason, various systems capable of relieving the stress of modern people are in the spotlight.

Modern people with limited time require good quality rest for stress care (or management). To this end, a relaxation method capable of remarkably improving stress or fatigue is being studied. For example, there has been proposed a method for finding a correlation between an external environment and the reduction in stress or fatigue and applying the found correlation to a rest environment.

### DISCLOSURE

### Technical Problem

The present disclosure may provide a therapy system for relieving stress and fatigue of a user, a bed system including the same, and an operating method of the therapy system. The present disclosure may provide a therapy system capable of creating an optimum environment for stress and fatigue relief based on a current state of a user, a bed system including the same, and an operating method of the therapy system.

### Technical Solution

A therapy system according to an embodiment of the present disclosure may include an electrocardiogram sensor that senses an electrocardiogram, a controller that determines a state step based on an R-R interval of the electrocardiogram and generates an oxygen control signal corresponding to the state step, and an oxygen supply device that releases oxygen having a concentration higher than an oxygen concentration in the atmosphere during an output time corresponding to the state step, based on the oxygen control signal. As an example, the oxygen supply device may release the oxygen having a target concentration corresponding to the state step, based on the oxygen control signal.

As an example, the controller may further generate a light control signal corresponding to the state step, and the therapy system may further include a lighting device that outputs a light during a lighting time corresponding to the state step, based on the light control signal. As an example, the lighting device may output a light of a wavelength corresponding to the state step, based on the light control signal. As an example, the lighting device may adjust a light output time corresponding to the state step, based on the light control signal.

As an example, the controller may further generate a sound control signal corresponding to the state step, and the therapy system may further include a speaker that outputs a sound corresponding to the state step, based on the sound control signal.

As an example, the electrocardiogram sensor may include a receive electrode that is spaced from a user and receives an electrocardiogram signal through capacitive coupling.

As an example, the controller may detect the R-R interval from the electrocardiogram, may extract a characteristic based on the R-R interval, and may select the state step corresponding to the characteristic from among a plurality of state steps. The state step may include a stress step and a fatigue step, and the controller may select a stress step corresponding to the characteristic from among a plurality of stress steps and may select a fatigue step corresponding to the characteristic from among a plurality of fatigue steps.

As an example, the electrocardiogram sensor may further sense an electrocardiogram changed while the oxygen is released, the controller may adjust the state step based on the changed electrocardiogram, and the oxygen supply device may adjust the output time based on the adjusted state step.

A bed system according to an embodiment of the present disclosure may include a body that includes a lower frame and an upper frame facing each other with respect to a use area, a sheet that is disposed on the lower frame and in which an electrocardiogram sensor sensing an electrocardiogram is embedded, a lighting device that is disposed on a first surface of the upper frame and outputs a light toward the use area, an oxygen supply device that is disposed on a second surface of the upper frame and releases oxygen toward the use area, a concentration of the oxygen being higher than an oxygen concentration in the atmosphere, and a controller. Based on the electrocardiogram, the controller may adjust an output time of the light and/or a wavelength of the light and may adjust an output time of the oxygen and/or the concentration of the oxygen.

As an example, the controller may select one state step of a plurality of state steps based on the electrocardiogram and may control the light and the oxygen based on the selected state step. As an example, the controller may detect an R-R interval from the electrocardiogram and may select the one state step in such a way that a characteristic extracted from the R-R interval corresponds to a characteristic range of each of the plurality of state steps.

As an example, the bed system may further include a display that is disposed on a third surface of the upper frame and outputs an image, and the controller may output state information to the display based on the electrocardiogram.

As an example, the electrocardiogram sensor may include a first electrode that is disposed in the sheet so as to be spaced from a user and receives a positive electrocardiogram signal through capacitive coupling, a second electrode that is disposed in the sheet so as to be spaced from the user and receives a negative electrocardiogram signal through capacitive coupling, and a differential amplifier that generates an amplification signal based on a difference between the positive electrocardiogram signal and the negative electrocardiogram signal.

An operating method of a therapy system according to an embodiment of the present disclosure may include measuring, by an electrocardiogram sensor, an electrocardiogram, determining, by a controller, a state step based on an R-R interval of the electrocardiogram, determining, by the controller, an oxygen output time of an oxygen supply device based on the state step, and releasing, by the oxygen supply device, oxygen having a concentration higher than an oxygen concentration in the atmosphere. As an example, the method may further include determining, by the controller, a target concentration of the oxygen based on the state step.

As an example, the method may further include measuring, by the electrocardiogram sensor, a change of the electrocardiogram, adjusting, by the controller, the state step based on an R-R interval of the changed electrocardiogram, and adjusting, by the controller, the oxygen output time or the target concentration of the oxygen based on the adjusted state step.

As an example, the method may further include determining, by the controller, at least one of a wavelength and an output time of a light to be output from a lighting device based on the state step, and outputting, by the lighting device, the light. As an example, the method may further include measuring, by the electrocardiogram sensor, a change of the electrocardiogram, adjusting, by the controller, the state step based on an R-R interval of the changed electrocardiogram, and adjusting, by the controller, at least one of the wavelength and the output time of the light based on the adjusted state step.

### Advantageous Effects

According to an embodiment of the present disclosure, a therapy system, a bed system including the same, and an operating method of the therapy system may provide an optimized stress or fatigue relief environment to a user by complexly controlling oxygen, a light, a sound, and the like based on an electrocardiogram of the user.

Also, according to the present disclosure, there may be no need to restrict the user for the purpose of providing an optimum therapy environment, and stress care may be performed in consideration of a difference between stress relief levels for respective individuals.

### DESCRIPTION OF DRAWINGS

FIG. 1 is a diagram illustrating a therapy system according to an embodiment of the present disclosure.

FIG. 2 is an exemplary diagram illustrating a bed system to which a therapy system of FIG. 1 is applied.

FIG. 3 is an exemplary circuit diagram illustrating an electrocardiogram sensor of FIG. 1 or 2.

FIG. 4 is an exemplary block diagram of a controller of FIG. 1.

FIG. 5 is an exemplary diagram for describing how a controller of FIG. 4 analyzes an electrocardiogram and determines a state step.

FIG. 6 is an exemplary flowchart of an operating method of a therapy system of FIG. 1.

FIG. 7 is an exemplary flowchart illustrating operation S120 of FIG. 6 in detail.

### BEST MODE

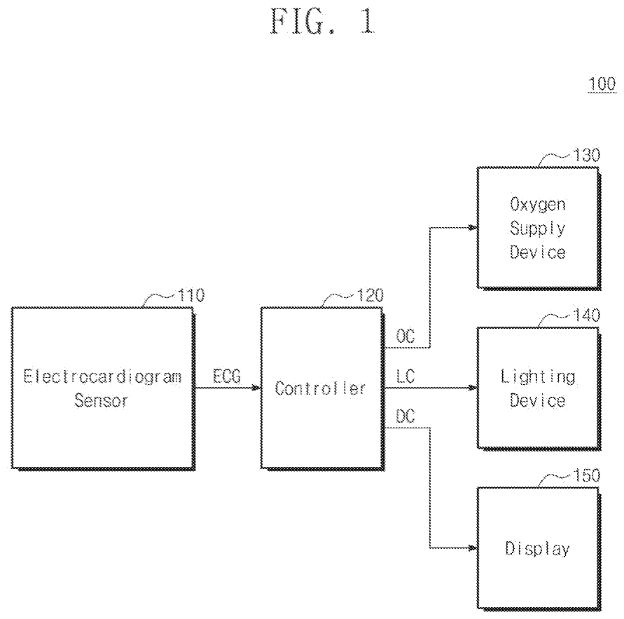

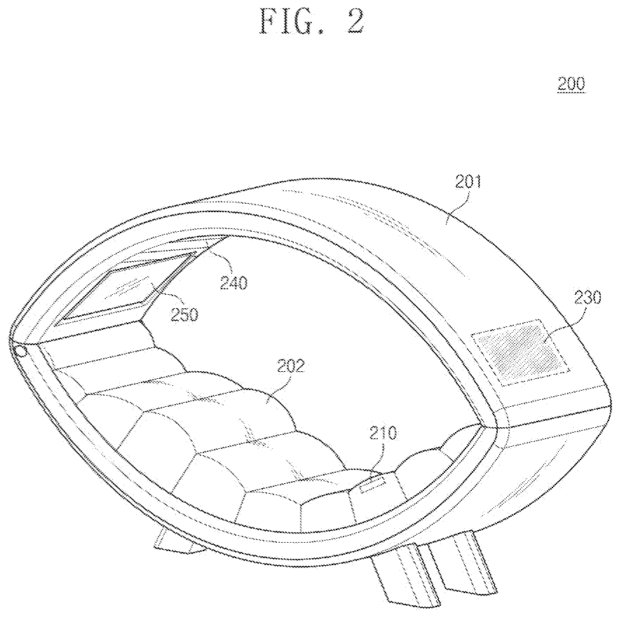

The best mode for embedding the present disclosure is a therapy system of FIG. 1, in which a complex therapy of oxygen, a light, a sound, and the like, which is capable of being implemented with a bed system of FIG. 2, is possible.

### Mode for Invention

Below, embodiments of the present disclosure will be described clearly and in detail with reference to accompanying drawings to such an extent that an ordinary one in the art implements embodiments of the present disclosure.

FIG. 1 is a diagram illustrating a therapy system according to an embodiment of the present disclosure. A therapy system 100 may be understood as a system that provides an environment for relieving stress or fatigue of a user based on biometric information of the user. Referring to FIG. 1, the therapy system 100 includes an electrocardiogram sensor 110, a controller 120, an oxygen supply device 130, a lighting device 140, and a display 150. The components included in the therapy system 100 may be understood as an example, and the therapy system 100 of the present disclosure is not limited to the example of FIG. 1.

The electrocardiogram sensor 110 may sense an electrocardiogram of the user. The electrocardiogram represents the electrical activity that occurs in the myocardium as the heart beats. The electrocardiogram sensor 110 may generate an electrocardiogram signal ECG corresponding to the electrocardiogram of the user. The electrocardiogram signal ECG may refer to information that is obtained by processing the electrical activity of the heart over time to an electrical signal capable of being analyzed. The electrocardiogram sensor 110 may generate the electrocardiogram signal ECG by measuring the electrocardiogram of the user and amplifying the measured electrocardiogram.

The electrocardiogram sensor 110 may not restrict the action or the like of the user for the purpose of sensing the electrocardiogram. The electrocardiogram sensor 110 may not force the user to conduct the following: an action of allowing a part of his/her body to make contact with a specific area thereof for the purpose of sensing the electrocardiogram. To this end, the electrocardiogram sensor 110 may be configured to sense the electrocardiogram in a state of being separated from the user without making contact with his/her skin. For example, the electrocardiogram sensor 110 may receive the electrocardiogram from the user through a receive electrode of a capacitive coupling manner. The receive electrode and the user may be capacitively coupled, with clothes or cushion interposed therebetween. The electrocardiogram may be transferred to the electrocardiogram sensor 110 through a capacitance between the user and the receive electrode. Accordingly, the electrocardiogram of the user may be measured without interfering with the purpose of stress relief of the therapy system 100.

The controller 120 may analyze the electrocardiogram signal ECG to evaluate a stress or fatigue level of the user. First, to process the electrocardiogram signal ECG, the controller 120 may perform pre-processing on the electrocardiogram signal ECG, that is, may remove or filter a noise of the electrocardiogram signal ECG. However, the pre-processing may be performed by using a filter or the like in the electrocardiogram sensor 110. The controller 120 may detect an R-peak value from the electrocardiogram signal ECG. The electrocardiogram may include P, Q, R, S, and T waves according to the heartbeat, and the electrocardiogram has a maximum value by the R wave. The R-peak value may be a maximum value of the electrocardiogram signal ECG corresponding to one heartbeat.

The controller 120 may detect an R-R interval being a time interval between R-peak values. The controller 120 may calculate the heart rate variability (HRV) indicating variability of heartbeats by using the R-R interval. For example, the heart rate variability may reflect an interaction of a sympathetic nerve and a parasympathetic nerve. Through the analysis of the heart rate variability, states of the sympathetic nerve and the parasympathetic nerve may be quantitatively evaluated. For example, an increase of the stress may cause an increase of heartbeat and an increase of systolic blood pressure, which is reflected to the heart rate variability.

The controller 120 may analyze the heart rate variability in at least one of a time domain and a frequency domain, and may extract characteristics for quantifying the stress or fatigue of the user. In the case of the time domain, the controller 120 may analyze the heart rate variability based on time intervals of Q, R, and S waves, and for example, SDNN, SDANN, RMSSD, NN50, pNN50, or the like may be utilized. In the case of the frequency domain, the controller 120 may detect a characteristic for each frequency band of the heart rate variability to evaluate a power value of the corresponding band. The above way to analyze the heart rate variability is an example, and the electrocardiogram analyzing way of the present disclosure is not limited thereto.

The controller 120 may evaluate the stress or fatigue level of the user based on the extracted characteristics. The controller 120 may calculate values of an evaluation index of stress or fatigue from the extracted characteristics. For example, the evaluation index may include a stress index and a fatigue index. For example, the controller 120 may allocate a weight corresponding to an evaluation index to each of the extracted characteristics and may calculate values of each of the stress index and the fatigue index. The evaluation index described above is provided as an example, and a stress or fatigue level evaluating way of the present disclosure is not limited thereto.

The controller 120 may determine a state step of stress or fatigue based on the values of the evaluation index thus calculated. For example, a state step may include a stress step and a fatigue step. The stress step or the fatigue step may be divided into a plurality of steps. The controller 120 may select one of a plurality of stress steps and one of a plurality of fatigue steps, based on the evaluation index. For example, the controller 120 may select a stress step based on a stress index and a fatigue index of the evaluation index. The controller 120 may provide stress care (or management) appropriate for the user by selecting a state step corresponding to an electrocardiogram of the user from among a plurality of state steps. Also, by providing a limited number of state steps, the amount of computation for the stress care may decrease.

The controller 120 may select one of the plurality of state steps. For example, electrocardiograms under various physical conditions, such as age, may be in advance collected to construct the database. Through the database, a correspondence relationship between stress and fatigue according to the electrocardiogram (or the R-R interval or the heart rate variability) may be in advance defined. Based on the defined correspondence relationship, the controller 120 may classify and manage characteristic ranges so as to correspond to the number of stress steps or fatigue steps. Afterwards, in the case where the electrocardiogram of the user is sensed, the controller 120 may select a state step based on a characteristic range to which a characteristic value of the user belongs. The above way to select a state step is provided as an example, and the present disclosure is not limited thereto.

The controller 120 may generate an oxygen control signal OC, a light control signal LC, and state information DC for display, based on the selected state step. The oxygen control signal OC may be a signal for determining at least one of a concentration and an output time of oxygen that is released from the oxygen supply device 130. The light control signal LC may be a signal for determining at least one of a wavelength and an output time of a light that is output from the lighting device 140. The state information DC may be image information for providing a stress and fatigue level corresponding to a state step to the user. That is, the therapy system 100 may analyze the sensed electrocardiogram to provide an optimized environment for relieving the user-specific stress and fatigue.

The oxygen supply device 130 releases oxygen toward the user based on the oxygen control signal OC. The oxygen thus released may have a concentration than an average oxygen concentration in the atmosphere. The oxygen supply device 130 may determine a concentration and an output time of oxygen based on the oxygen control signal OC. For example, as a stress or fatigue level indicated by a state step increases, a concentration of oxygen to be released may become higher, and an output time of oxygen may become longer. That is, the oxygen supply device 130 may provide an optimum oxygen therapy in consideration of a current state of the user.

Sufficient oxygen supply may make up for oxygen deficiency due to stress. Also, the sufficient oxygen supply may reduce a hormone occurring due to the stress. In a recovery period after the stress occurs, when oxygen is provided, the parasympathetic nerve may be more dominant than the sympathetic nerve. In addition, oxygen has medical advantages such as blood circulation, tissue regeneration, detoxification, blood pressure regulation, and energy supply to cell. Also oxygen provides the following positive advantages: increasing of thinking, memory, and concentration due to enhanced cerebral activity; strengthening of the body's resistance; stress/fatigue relief; skin beauty; odor removal; resolving of oxygen deficiency; creating of a comfortable indoor environment. That is, the therapy system 100 may care the stress and fatigue of the user through the oxygen supply device 130.

The lighting device 140 outputs a light toward the user based on the light control signal LC. The lighting device 140 may determine a wavelength and an output time of a light based on the light control signal LC. The lighting device 140 may output a light with a wavelength of a specific color during an output time corresponding to a state step that is selected as a result of analyzing the sensed electrocardiogram. For example, as a stress or fatigue level indicated by a state step increases, an output time of a light may become longer. That is, the lighting device 140 may provide an optimum light therapy in consideration of a current state of the user.

The lighting device 140 may output a light whose light source color corresponds to a state step. Light source colors may cause different responses of the autonomic nervous system. The lighting device 140 may relieve the stress and fatigue by providing a light source color appropriate for a current state of the user. For example, a red light may activate the user's brain, may promote blood circulation, and may allow a skin to whiten or skin elasticity to increase. For example, an orange light may alleviate anxiety of the user and may reduce a skin conduction response (SCR) and a heartbeat. For example, a yellow light may remove mucus secretions caused by a cold or like and may increase the memory. For example, a green light may act on the sympathetic nerve to induce balanced mind. For example, a cyan light may induce psychological arousal so as to alleviate the user's sad emotions and may increase the skin conduction response. For example, a blue light may induce calm emotions and may promote recovery after stress.

The display 150 may display an image corresponding to the state information DC. The state information DC may visually inform current stress or fatigue of the user. For example, the display 150 may display values of a current evaluation index, whether a therapy is required, a kind of a therapy to be provided, stress or fatigue relief, and the like.

Although not illustrated in FIG. 1, the therapy system 100 may further provide a sound therapy based on the electrocardiogram. To this end, the therapy system 100 may further include a speaker (not illustrated) that outputs a sound corresponding to a state step. In this case, the controller 120 may generate a sound control signal based on the selected state step, and the speaker (not illustrated) may output a sound corresponding to a state step based on the sound control signal. For example, the speaker (not illustrated) may determine a wavelength, an intensity, and an output time of a sound based on the sound control signal.

The therapy system 100 may sense the electrocardiogram of the user continuously or periodically during the therapy, may adjust a state step, and may change oxygen, a light, a sound, and an image to be displayed so as to be appropriate for the adjusted state step. For example, in the case that a result of analyzing the electrocardiogram of the user indicates that the stress is relieved, the controller 120 may decrease an output time of oxygen, a concentration of oxygen, or an output time of a light, or may change a color. That is, the therapy system 100 may provide a complex therapy (or multi-therapy) to the user by reflecting a current state of the user continuously or periodically.

The therapy system 100 may separately manage a therapy history of the user. For example, the controller 120 may store and manage identification information of the user, and may store and manage a previous therapy history or pattern of a user corresponding to the identification information. In the case where the user is again provided with the therapy later, the controller 120 may adjust oxygen, a light, or a sound in consideration of the therapy history or pattern. For example, an oxygen supply time, an oxygen concentration, and the like, which are associated with the relief of an electrocardiogram signal of a previous user may be recorded at the therapy system 100 to construct a pattern, and an oxygen supply time and an oxygen concentration may be determined to correspond to the pattern.

FIG. 2 is a diagram illustrating a bed system to which a therapy system of FIG. 1 is applied. A bed system 200 provides a use area where the user may recline or lie down to take a break. Because the bed system 200 includes the components of the therapy system 100 of FIG. 1, the bed system 200 may provide an environment for relieving the stress or fatigue of the user based on biometric information of the user. Referring to FIG. 2, the bed system 200 includes a body 201, a sheet 202, an electrocardiogram sensor 210, an oxygen supply device 230, a lighting device 240, and a display 250.

The electrocardiogram sensor 210, the oxygen supply device 230, the lighting device 240, and the display 250 of FIG. 2 may correspond to the electrocardiogram sensor 110, the oxygen supply device 130, the lighting device 140, and the display 150, respectively. Although not illustrated in FIG. 2, a component corresponding to the controller 120 of FIG. 1 may be provided in the bed system 200. Also, the bed system 200 may further include a speaker (not illustrated) for providing a sound therapy based on an electrocardiogram.

The body 201 may include a lower frame on which the sheet 202 is disposed, and an upper frame on which the oxygen supply device 230, the lighting device 240, the display 250, and the like are disposed. The upper frame and the lower frame may be connected with each other, and the use area for user's relaxation may be provided between the lower frame and the upper frame. In an embodiment, the body 201 is illustrated as surrounding the use area, but a shape of the body 201 is not limited thereto.

The sheet 202 may be disposed on the lower frame of the body 201. The sheet 202 may include a member, such as a cushion, such that the user may lie down or recline. The electrocardiogram sensor 210 may be embedded in the sheet 202 so as to sense an electrocardiogram of the user. The electrocardiogram sensor 210 may be spaced from the user to sense the electrocardiogram in a capacitive coupling manner. Accordingly, because there is no need for the user to make direct contact with the electrocardiogram sensor 210, the electrocardiogram may be sensed in a state where an action of the user is not restricted.

The electrocardiogram sensor 210 is illustrated in FIG. 2 as being disposed within the sheet 202, but the present disclosure is not limited thereto. For example, at least part (e.g., a receive electrode) of the electrocardiogram sensor 210 may be exposed on a surface of the sheet 202. Also, the electrocardiogram sensor 210 is illustrated in FIG. 2 as being disposed close to the user's heart, but the present disclosure is not limited thereto. For example, at least part of the electrocardiogram sensor 210 may be disposed to sense the electrocardiogram by using differential signals or may be disposed in any other area of the sheet 202 for ground implementation.

The oxygen supply device 230 may be disposed in/on the body 201, and for example, the oxygen supply device 230 may be disposed on a first surface of the upper frame. To supply oxygen effectively, the oxygen supply device 230 may be disposed adjacent to the user's head. The oxygen supply device 230 may release oxygen toward the user based on the electrocardiogram sensed from the electrocardiogram sensor 210. A concentration and/or an output time of oxygen may be determined based on the electrocardiogram.

The lighting device 240 may be disposed in/on the body 201, and for example, the lighting device 240 may be disposed on a second surface of the upper frame. The lighting device 240 may be disposed on a central portion of the upper frame such that a light is effectively provided without providing excessive visual stimulation to the user. The lighting device 240 may release oxygen toward the user based on the electrocardiogram sensed from the electrocardiogram sensor 210. A wavelength and/or an output time of a light may be determined based on the electrocardiogram.

The display 250 may be disposed in/on the body 201, and for example, the display 250 may be disposed on a third surface of the upper frame. The display 250 may be disposed to face a view direction of the user such that the user views an image effectively. That is, the oxygen supply device 230, the lighting device 240, and the lighting device 240 may be disposed in order along the upper frame with respect to the user's head. The display 250 may display the following information: an evaluation index and a state step evaluated depending on the electrocardiogram sensed from the electrocardiogram sensor 210.

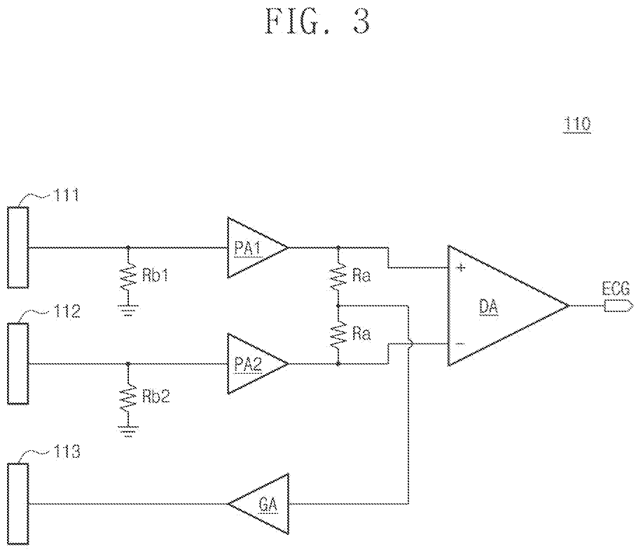

FIG. 3 is an exemplary circuit diagram illustrating an electrocardiogram sensor of FIG. 1 or 2. The electrocardiogram sensor 110 of FIG. 3 corresponds to the electrocardiogram sensor 110 of FIG. 1 or the electrocardiogram sensor 210 of FIG. 2. Referring to FIG. 3, the electrocardiogram sensor 110 may include first to third electrodes 111 to 113, first and second pre-amplifiers PA1 and PA2, a gain amplifier GA, and a differential amplifier DA. The components included in the electrocardiogram sensor 110 may be provided as an example, and the electrocardiogram sensor 110 of the present disclosure is not limited to the example of FIG. 3. For example, the electrocardiogram sensor 110 may further include a converter for converting an analog electrocardiogram signal into a digital signal or may further include a filter or a pre-processing circuit for removing a noise.

The first electrode 111 may receive a positive electrocardiogram signal in a capacitive coupling manner. The first electrode 111 may be disposed close to the user's heart, and for example, may be disposed in/on the sheet 202 of FIG. 2. The first electrode 111 may not make direct contact with the user's skin and may be a dry electrode. The first electrode 111 and the user may form a capacitor, with a fabric interposed therebetween. The positive electrocardiogram signal that is generated depending on a heartbeat may be transferred to the electrocardiogram sensor 110 through the capacitor. The positive electrocardiogram signal may be input to the first pre-amplifier PA1 as a voltage signal by a first resistor Rb1.

The second electrode 112 may receive a negative electrocardiogram signal in the capacitive coupling manner. The second electrode 112 may be disposed to be spaced from the user's heart compared to the first electrode 111; for example, the second electrode 112 may be disposed on/in the sheet 202 of FIG. 2 so as to be adjacent to his/her right breast. The second electrode 112 may not make direct contact with the user's skin and may be a dry electrode. The second electrode 112 and the user may form a capacitor, with a fabric interposed therebetween. The negative electrocardiogram signal may be transferred to the electrocardiogram sensor 110 through the capacitor. The negative electrocardiogram signal may be input to the second pre-amplifier PA2 as a voltage signal by a second resistor Rb2.

The first pre-amplifier PA1 may amplify the positive electrocardiogram signal. The amplified positive electrocardiogram signal may be input to a positive input terminal of the differential amplifier DA. The second pre-amplifier PA2 may amplify the negative electrocardiogram signal. The amplified negative electrocardiogram signal may be input to a negative input terminal of the differential amplifier DA.

A medium voltage of the amplified positive electrocardiogram signal and the amplified negative electrocardiogram signal may be input to the gain amplifier GA by third resistors Ra. A signal amplified by the gain amplifier GA may be output to the third electrode 113. The third electrode 113 may be capacitively coupled with the user. The third electrode 113 may be disposed distant from the user's heart, and for example, may be disposed in/on the sheet 202 of FIG. 2. For example, the third electrode 113 may be disposed adjacent to the user's right leg. The third electrode 113 may not make direct contact with the user's skin and may be a dry electrode. The third electrode 113 may be used to form a ground of the electrocardiogram sensor 110.

The differential amplifier DA may generate an amplification signal by amplifying a difference between the amplified positive electrocardiogram signal and the amplified negative electrocardiogram signal. The amplification signal may be the electrocardiogram signal ECG. As described above, the electrocardiogram sensor 110 may remove a nose of the electrocardiogram signal ECG or may perform pre-processing on the electrocardiogram signal ECG so as to be analyzed by the controller 120. The electrocardiogram sensor 110 may measure the electrocardiogram in the capacitive coupling manner, thus not restricting the user.

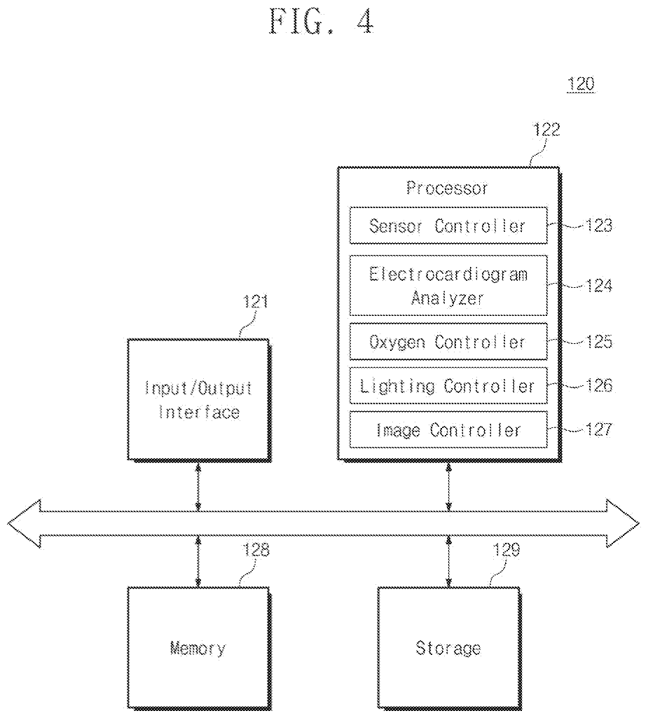

FIG. 4 is an exemplary block diagram of a controller of FIG. 1. The controller 120 of FIG. 4 corresponds to the controller 120 of FIG. 1. The controller 120 of FIG. 4 may be included in the bed system 200 of FIG. 2. Referring to FIG. 4, the controller 120 includes an input/output interface 121, a processor 122, a memory 128, and storage 129. The input/output interface 121, the processor 122, the memory 128, and the storage 129 may exchange data with each other through a bus. The components included in the controller 120 are provided as an example, and the controller 120 of the present disclosure is not limited to the example of FIG. 4. For convenience of description, FIG. 4 will be described with reference to reference numerals/marks of FIG. 1.

The input/output interface 121 is configured to receive the electrocardiogram signal ECG from the electrocardiogram sensor 110 of FIG. 1 and to output the oxygen control signal OC, the light control signal LC, and the state information DC to the oxygen supply device 130, the lighting device 140, and the display 150, respectively. The input/output interface 121 may provide the received electrocardiogram signal ECG to the processor 122, the memory 128, and the storage 129 through the bus.

The processor 122 may function as a central processing unit of the therapy system 100 or the controller 120. The processor 122 may analyze the electrocardiogram signal ECG to perform a control operation and a computation/calculation operation necessary to control an image. For example, under control of the processor 122, the input/output interface 121 may receive the electrocardiogram signal ECG. A computation/calculation operation for analyzing the electrocardiogram signal ECG, evaluating a state step, and generating the oxygen control signal OC, the light control signal LC, and the state information DC may be performed under control of the processor 122.

The processor 122 may include a sensor controller 123, an electrocardiogram analyzer 124, an oxygen controller 125, a lighting controller 126, and an image controller 127. Each component of the processor 122 may operate by utilizing a computation/calculation space of the memory 128 and may read files for driving an operating system and execution files of applications from the storage 129. The processor 122 may execute the operating system and the applications.

The sensor controller 123, the electrocardiogram analyzer 124, the oxygen controller 125, the lighting controller 126, and the image controller 127 may be implemented by software or firmware. In this case, the firmware may be stored in the storage 129 and may be loaded onto the memory 128 in executing the firmware. The processor 122 may execute the firmware loaded onto the memory 128. However, the present disclosure is not limited thereto. For example, the sensor controller 123, the electrocardiogram analyzer 124, the oxygen controller 125, the lighting controller 126, and the image controller 127 may be implemented with a dedicated logic circuit such as a FPGA (Field Programmable Gate Array) or ASIC (Application Specific Integrated Circuit).

The sensor controller 123 may control an operation of the electrocardiogram sensor 110 of FIG. 1. Under control of the sensor controller 123, the electrocardiogram sensor 110 may sense an electrocardiogram of the user before providing the user with a therapy. For example, the sensor controller 123 may generate a signal of activating the electrocardiogram sensor 110 at a given time. Here, the given time may be a time before the therapy is provided, and the present disclosure is not limited thereto. For example, the given time may be a continuous time having a specific period. In the case where the electrocardiogram sensor 110 continuously senses the electrocardiogram of the user, oxygen, a light, and an image may be adaptively adjusted depending on a change of the electrocardiogram.

The electrocardiogram analyzer 124 may analyze the received electrocardiogram signal ECG and may evaluate a state step. As described with reference to FIG. 1, the electrocardiogram analyzer 124 may detect an R-peak value and an R-R interval from the electrocardiogram signal ECG. The electrocardiogram analyzer 124 may calculate the heart rate variability through the R-R interval. The electrocardiogram analyzer 124 may extract characteristics for quantifying stress or fatigue from the heart rate variability. The electrocardiogram analyzer 124 may calculate values of an evaluation index of stress or fatigue from the extracted characteristics. The electrocardiogram analyzer 124 may determine a state step of stress or fatigue based on the values of the evaluation index thus calculated.

The oxygen controller 125 may generate the oxygen control signal OC corresponding to the state step determined by the electrocardiogram analyzer 124. The oxygen control signal OC may be used to control a concentration and an output time of oxygen that is released from the oxygen supply device 130.

The lighting controller 126 may generate the light control signal LC corresponding to the state step determined by the electrocardiogram analyzer 124. The light control signal LC may be used to control a wavelength and an output time of a light that is output from the lighting device 140.

The image controller 127 may generate the state information DC corresponding to the state step determined by the electrocardiogram analyzer 124. The state information DC may include image information indicating a current stress or fatigue level of the user.

Although not illustrated, the processor 122 may further include a sound controller. The sound controller may generate the sound control signal corresponding to the state step determined by the electrocardiogram analyzer 124. The sound control signal may be used to control a sound that is output through the speaker or the like.

The memory 128 may store data and program codes that are processed by the processor 122 or are scheduled to be processed by the processor 120. For example, the memory 128 may store information, which is generated in the process of analyzing the electrocardiogram signal ECG, such as the electrocardiogram signal ECG, an R-R interval, heart rate variability information, values of an evaluation index, and state step information provided from the input/output interface 121, and information necessary to analyze the electrocardiogram signal ECG. The memory 128 may be used as a main memory of the therapy system 100 or the controller 120.

The storage 129 may store data generated for the purpose of long-time storage by the operating system or the applications, files for driving the operating system, execution files of the applications, etc. For example, the storage 129 may store files for executing the sensor controller 123, the electrocardiogram analyzer 124, the oxygen controller 125, the lighting controller 126, and the image controller 127. The storage 129 may be used as an auxiliary memory of the therapy system 100 or the controller 120.

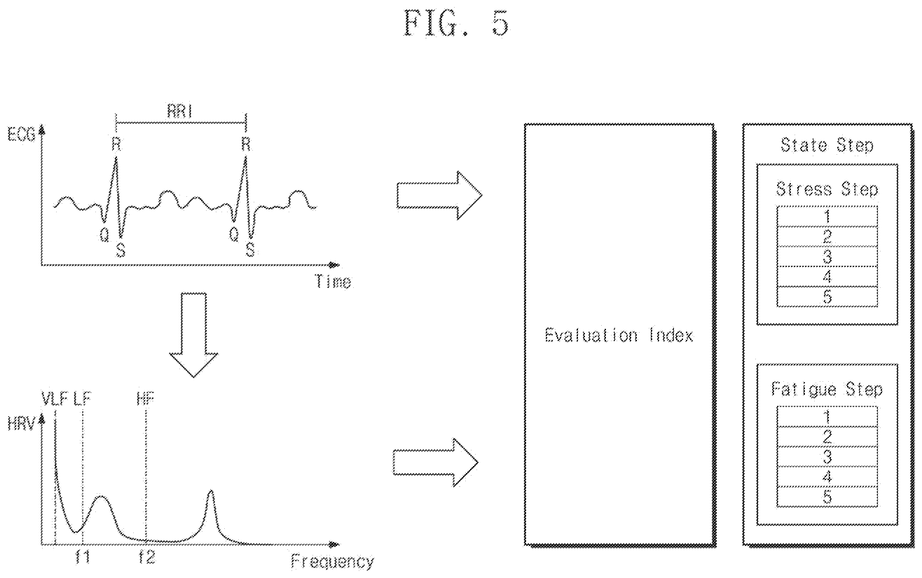

FIG. 5 is an exemplary diagram for describing how a controller of FIG. 4 analyzes an electrocardiogram and determines a state step. Referring to FIG. 5, the electrocardiogram signal ECG over time may be provided from the electrocardiogram sensor 110 of FIG. 1 to the controller 120. For convenience of description, FIG. 5 will be described with reference to reference numerals/marks of FIG. 4.

The electrocardiogram signal ECG includes the Q wave, the R wave, and the S wave. The Q wave refers to the downward or negative deflection in which an electrical activity current of the heart decreases The R wave refers to the upward or positive deflection in which an electrical activity current of the heart increases after the Q wave. The electrocardiogram may have a maximum value by the R wave, and the maximum value is defined as an R-peak value. The S wave refers to the downward or negative deflection in which an electrical activity current of the heart decreases after the R wave The Q, R, and S waves come from a depolarization process of the ventricular muscle.

The electrocardiogram analyzer 124 may extract an R-peak value from the electrocardiogram signal ECG. The electrocardiogram analyzer 124 may detect an R-R interval RRI being a time interval between R-peak values. The controller 120 may calculate the heart rate variability HRV based on the R-R interval RRI. The heart rate variability HRV may appear in a time domain or a frequency domain, and for example, a power spectral density PSD in the frequency domain is illustrated as a waveform of the heart rate variability HRV.

The electrocardiogram analyzer 124 may extract characteristics for quantifying stress or fatigue of the user. For example, the electrocardiogram analyzer 124 may classify the heart rate variability as a high frequency band HF, a low frequency band LF, or a very low frequency band VLF for the frequency domain. For example, the low frequency band LF and the very low frequency band VLF may be distinguished based on a first frequency f1, and the low frequency band LF and the high frequency band HF may be distinguished based on a second frequency f2. For example, a power ratio LF/HF of the low frequency band LF to the high frequency band HF may be extracted as a characteristic. An increase of the power ratio LF/HF may indicate that the sympathetic nerve is activated or the activity of the parasympathetic nerve is suppressed. In addition, the electrocardiogram analyzer 124 may extract characteristics in the time domain or may extract characteristics from the electrocardiogram signal ECG itself.

The electrocardiogram analyzer 124 may evaluate a stress or fatigue level of the user based on the extracted characteristics. The electrocardiogram analyzer 124 may calculate values of an evaluation index of stress or fatigue from the extracted characteristics. For example, the evaluation index may include a stress index, autonomic balance, autonomic activity, stress resistance, and fatigue, and values respectively corresponding to indexes may be calculated.

The electrocardiogram analyzer 124 may determine a state step based on the values of the evaluation index thus calculated. For example, a state step may include a stress step and a fatigue step. The stress step or the fatigue step may be divided into a plurality of steps. The state step may be divided into a plurality of steps from a low level to a high level of stress or fatigue. The electrocardiogram analyzer 124 may select one of a plurality of stress steps and one of a plurality of fatigue steps, based on the evaluation index. For example, the electrocardiogram analyzer 124 may select a stress step based on a stress index and a fatigue index of the evaluation index.

The oxygen controller 125 may control a concentration or an output time of oxygen, which is output from the oxygen supply device 130, based on the selected state step. In the case where each of the stress step and the fatigue step is divided into 5 steps, the oxygen controller 125 may control the oxygen supply device 130 in the number of up to 25 cases.

The lighting controller 126 may control a wavelength or an output time of a light, which is output from the lighting controller 126, based on the selected state step. In the case where each of the stress step and the fatigue step is divided into 5 steps, the lighting controller 126 may control the lighting device 140 in the number of up to 25 cases.

The image controller 127 may display an image corresponding to the selected state step. For example, the display 150 may display 5 evaluation indexes in a pentagonal graph and may display a current stress step and a current fatigue step. The display 150 may display an oxygen concentration, an oxygen output time, a light color, or a light output time according to a state step. In addition, when the electrocardiogram of the user changes depending on the therapy, the display 150 may display the changed evaluation index values and state step. Also, changed therapy information may be further displayed.

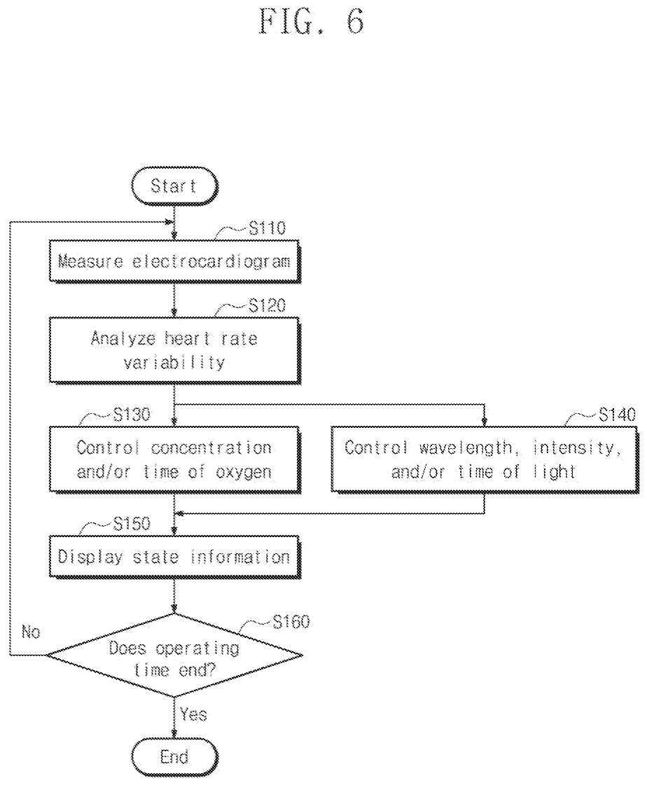

FIG. 6 is an exemplary flowchart of an operating method of a therapy system of FIG. 1. Operations of FIG. 6 may be performed by the therapy system 100 described with reference to FIG. 1 or the bed system 200 of FIG. 2 including the therapy system 100. For convenience of description, FIG. 6 will be described with reference to reference marks/numerals of FIG. 1.

In operation S110, the electrocardiogram sensor 110 may measure an electrocardiogram of the user. The electrocardiogram sensor 110 may receive the electrocardiogram in the capacitive coupling manner such that an action of the user is not restricted.

In operation S120, the controller 120 may analyze the electrocardiogram measured by the electrocardiogram sensor 110. For example, the controller 120 may detect an R-peak value from the electrocardiogram and may an R-R interval. The controller 120 may calculate the heart rate variability through the R-R interval and may extract characteristics for quantifying stress or fatigue from the heart rate variability. The controller 120 may calculate values of an evaluation index of stress or fatigue from the extracted characteristics and may determine a state step of the stress or fatigue.

In operation S130, the controller 120 may control a concentration and an output time of oxygen that is released from the oxygen supply device 130. The controller 120 may generate the oxygen control signal OC corresponding to the state step determined in operation S120. The oxygen supply device 130 may release air of a target oxygen concentration during the output time corresponding to the state step, based on the oxygen control signal OC.

In operation S140, the controller 120 may control a wavelength and an output time of a light that is output from the lighting device 140. The controller 120 may generate the light control signal LC corresponding to the state step determined in operation S120. The lighting device 140 may output a light of a target color during the output time corresponding to the state step, based on the light control signal LC.

In operation S150, the controller 120 may output the state information DC corresponding to the state step to the display 150. The display 150 may display the state information DC including the state step, the evaluation index, or therapy information.

Although not illustrated, the controller 120 may further generate the sound control signal corresponding to the state step. In this case, the speaker may output a sound corresponding to the state step based on the sound control signal.

In operation S160, whether an operating time ends is determined. The operating time may refer to a time during which a complex therapy is provided to the user. The operating time may depend on the output time of the oxygen controlled in operation S130 or the output time of the light controlled in operation S140. When it is determined that the operating time does not end, operation S110 to operation S150 may again be performed. The electrocardiogram of the user may change depending on therapy results of operation S130 and operation S140. The changed electrocardiogram may be measured in operation S110, and the state step may be adjusted depending on an analysis result thereof. In this case, a concentration and/or an output time of oxygen may be adjusted, and a wavelength, an intensity, and/or an output time of a light may be adjusted.

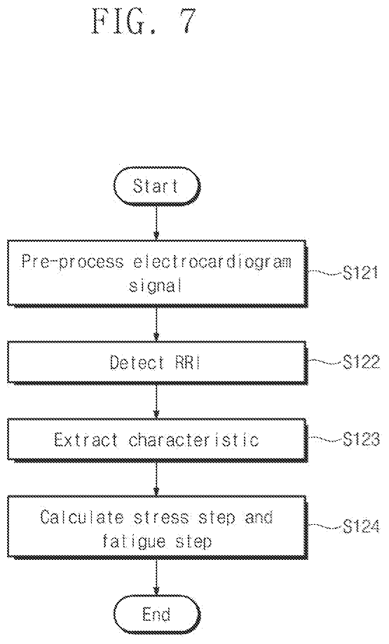

FIG. 7 is an exemplary flowchart illustrating operation S120 of FIG. 6 in detail. Operations of FIG. 7 may be performed by the controller 120 of FIG. 1. In operation S121, the controller 120 may pre-process the electrocardiogram received from the electrocardiogram sensor 110. For example, the controller 120 may remove a noise of the electrocardiogram signal ECG.

In operation S122, the controller 120 may detect an R-R-R interval from the pre-processed electrocardiogram signal ECG. The controller 120 may detect an R-peak value from the electrocardiogram signal ECG. The controller 120 may detect an R-R interval being a time interval between R-peak values.

In operation S123, the controller 120 may extract characteristics for quantifying stress or fatigue based on the R-R interval. For example, the controller 120 may calculate the heart rate variability indicating fluctuations in heartbeats based on the R-R interval. The controller 120 may extract the characteristics by analyzing the heart rate variability in at least one of the time domain and the frequency domain.

In operation S124, the controller 120 may calculate a stress step and a fatigue step based on the extracted characteristics. For example, the controller 120 may calculate values of an evaluation index of stress or fatigue from the extracted characteristics. The controller 120 may determine a state step of the stress or fatigue based on the values of the evaluation index thus calculated. The state step may be used to control the oxygen supply device 130, the lighting device 140, and the like.

The above-mentioned description refers to embodiments for implementing the scope of the present disclosure. Embodiments in which a design is changed simply or which are easily changed may be included in the scope of the present disclosure as well as an embodiment described above. In addition, technologies that are easily changed and implemented by using the above-mentioned embodiments may be also included in the scope of the present disclosure.

### INDUSTRIAL APPLICABILITY

The present disclosure may relate to a therapy system for stress care, a bed system including the same, and an operating method of the therapy system, and may provide an optimized stress or fatigue relief environment to a user by complexly controlling oxygen, a light, a sound, and the like based on an electrocardiogram of the user.

1. A therapy system comprising:
an electrocardiogram sensor configured to sense an electrocardiogram;
a controller configured to determine a state step based on an R-R interval of the electrocardiogram and to generate an oxygen control signal corresponding to the state step; and
an oxygen supply device configured to release oxygen having a concentration higher than an oxygen concentration in the atmosphere during an output time corresponding to the state step, based on the oxygen control signal.

2. The therapy system of claim 1, wherein the oxygen supply device releases the oxygen having a target concentration corresponding to the state step, based on the oxygen control signal.

3. The therapy system of claim 1, wherein the controller further generates a light control signal corresponding to the state step, and
further comprising:
a lighting device configured to output a light during a lighting time corresponding to the state step, based on the light control signal.

4. The therapy system of claim 1, wherein the controller further generates a light control signal corresponding to the state step, and
further comprising:
a lighting device configured to output a light of a wavelength corresponding to the state step, based on the light control signal.

5. The therapy system of claim 1, wherein the controller further generates a light control signal corresponding to the state step, and
further comprising:
a lighting device configured to output a light during an light output time corresponding to the state step, based on the light control signal.

6. The therapy system of claim 1, wherein the controller further generates a sound control signal corresponding to the state step, and
further comprising:
a speaker configured to output a sound corresponding to the state step, based on the sound control signal.

7. The therapy system of claim 1, wherein the electrocardiogram sensor includes:
a receive electrode spaced from a user, and configured to receive an electrocardiogram signal through capacitive coupling.

8. The therapy system of claim 1, wherein the controller detects the R-R interval from the electrocardiogram, extracts a characteristic based on the R-R interval, and selects the state step corresponding to the characteristic from among a plurality of state steps.

9. The therapy system of claim 8, wherein the state step includes a stress step and a fatigue step, and
wherein the controller selects a stress step corresponding to the characteristic from among a plurality of stress steps and selects a fatigue step corresponding to the characteristic from among a plurality of fatigue steps.

10. The therapy system of claim 1, wherein the electrocardiogram sensor further senses an electrocardiogram changed while the oxygen is released,
wherein the controller adjusts the state step based on the changed electrocardiogram, and
wherein the oxygen supply device adjusts the output time based on the adjusted state step.

11. A bed system comprising:
a body including a lower frame and an upper frame facing each other with respect to a use area;
a sheet disposed on the lower frame, wherein an electrocardiogram sensor sensing an electrocardiogram is embedded in the sheet;
a lighting device disposed on a first surface of the upper frame, and configured to output a light toward the use area;
an oxygen supply device disposed on a second surface of the upper frame, and configured to release oxygen toward the use area, a concentration of the oxygen being higher than an oxygen concentration in the atmosphere; and
a controller,
wherein, based on the electrocardiogram, the controller adjusts an output time of the light and/or a wavelength of the light and adjusts an output time of the oxygen and/or the concentration of the oxygen.

12. The bed system of claim 11, wherein the controller selects one state step of a plurality of state steps based on the electrocardiogram and controls the light and the oxygen based on the selected state step.

13. The bed system of claim 12, wherein the controller detects an R-R interval from the electrocardiogram and selects the one state step by matching a characteristic extracted from the R-R interval to a characteristic range of each of the plurality of state steps.

14. The bed system of claim 11, further comprising:
a display disposed on a third surface of the upper frame, and configured to output an image,
wherein the controller outputs state information to the display based on the electrocardiogram.

15. The bed system of claim 13, wherein the electrocardiogram sensor includes:
a first electrode disposed in the sheet so as to be spaced from a user, and configured to receive a positive electrocardiogram signal through capacitive coupling;
a second electrode disposed in the sheet so as to be spaced from the user, and configured to receive a negative electrocardiogram signal through capacitive coupling; and
a differential amplifier configured to generate an amplification signal based on a difference between the positive electrocardiogram signal and the negative electrocardiogram signal.

16. An operating method of a therapy system, the method comprising:
measuring, by an electrocardiogram sensor, an electrocardiogram;
determining, by a controller, a state step based on an R-R interval of the electrocardiogram;
determining, by the controller, an oxygen output time of an oxygen supply device based on the state step; and
releasing, by the oxygen supply device, oxygen having a concentration higher than an oxygen concentration in the atmosphere.

17. The method of claim 16, further comprising:
determining, by the controller, a target concentration of the oxygen based on the state step.

18. The method of claim 17, further comprising:
measuring, by the electrocardiogram sensor, a change of the electrocardiogram;
adjusting, by the controller, the state step based on an R-R interval of the changed electrocardiogram; and
adjusting, by the controller, the oxygen output time or the target concentration of the oxygen based on the adjusted state step.

19. The method of claim 16, further comprising:
determining, by the controller, at least one of a wavelength and an output time of a light to be output from a lighting device based on the state step; and
outputting, by the lighting device, the light.

20. The method of claim 19, further comprising:
measuring, by the electrocardiogram sensor, a change of the electrocardiogram;
adjusting, by the controller, the state step based on an R-R interval of the changed electrocardiogram; and
adjusting, by the controller, at least one of the wavelength and the output time of the light based on the adjusted state step.
